(19) United States
(12) Patent Application Publication
(10) Pub. No.: US 2006/0051825 A1
(43) Pub. Date: Mar. 9, 2006

(54) Methods and compositions for measuring canine BNP and uses thereof

(76) Inventors: Kenneth F. Buechler, Rancho Santa Fe, CA (US); Joseph Buechler, Carlsbad, CA (US)

Correspondence Address: Barry S. Wilson;Foley & Lardner LLP, P.O. Box 80278, San Diego, CA, 92138-0278, US

(21) Appl. No.: 11/222,494

(22) Filed: Sep. 7, 2005

Related U.S. Application Data
(60) Provisional application No. 60/609,015, filed on Sep. 9, 2004.

(51) Int. Cl. G01N 33/53; G01N 33/537; G01N 33/543

(52) U.S. Cl. 435/7.93

(57) ABSTRACT

The present invention describes compositions and methods designed to determine the presence or amount of BNP or fragments thereof in a sample. In particular, the invention provides materials that may be configured to bind canine BNP in a sandwich assay format. The present invention provides, inter alia, assays designed to rapidly and accurately measure BNP-related species in non-human animals.

### CROSS-REFERENCE RELATED TO PATENT APPLICATIONS

This application is claiming the benefit under 35 USC 119(e) of U.S. Application 60/609,015, filed Sep. 9, 2004, incorporated herein by reference in its entirety.

### FIELD OF THE INVENTION

The present invention relates to veterinary diagnostics.

### BACKGROUND OF THE INVENTION

The following discussion of the background of the invention is merely provided to aid the reader in understanding the invention and is not admitted to describe or constitute prior art to the present invention.

Natriuretic peptides are a group of naturally occurring substances that act in the body to oppose the activity of the renin-angiotensin system. There are three major natriuretic peptides: atrial natriuretic peptide (ANP), which is synthesized in the atria; brain-type natriuretic peptide (BNP), which is synthesized in the ventricles; and C-type natriuretic peptide (CNP), which is synthesized in the brain.

Mature human B-type natriuretic peptide (BNP) (also called brain-type natriuretic peptide and, in humans, BNP₇₇₋₁₀₈) is a biologically active peptide that is involved in the natriuresis system to regulate blood pressure and fluid balance (Bonow, R. O., Circulation 93:1946-1950, 1996). The mature human BNP hormone is generated by proteolytic cleavage of a 108-amino acid precursor molecule, referred to herein as “pro-BNP” (or BNP₁₋₁₀₈). Cleavage generates a 76-amino acid N-terminal peptide, referred to as “NT pro BNP” (or BNP₁₋₇₆) and the 32-amino acid mature BNP₇₇₋₁₀₈ hormone. It has been suggested that each of these species—NT pro-BNP, BNP-32, and the pre-pro-BNP—can circulate in human plasma (Tateyama et al., Biochem. Biophys: Res. Commun. 185:760-7, 1992; Hunt et al., Biochem. Biophys. Res. Commun. 214:1175-83, 1995). Similar polypeptides have been identified in numerous species, including pigs (Sus scrofa), cows (Bos Taurus), domestic dogs (Canis familiaris), domestic cats (Felis catus), sheep (Ovis aries), mice (Mus musculus), rats (Rattus norvegicus), etc. See, e.g., Liu et al., Gene 292: 183-190, 2002.

BNP is released in response to ventricular stretch, and will cause vasorelaxation, inhibition of aldosterone secretion in the adrenal cortex, and inhibition of renin secretion in the kidney. BNP release will cause natriuresis and a reduction in intravascular volume, effects amplified by the antagonism of antidiuretic hormone (ADH). Increased blood levels of BNP have been found in certain disease states, suggesting a role in the pathophysiology of those diseases, including stroke, congestive heart failure (CHF), cardiac ischemia, systemic hypertension, and acute myocardial infarction. See, e.g., WO 02/089657; WO 02/083913; and WO 03/016910, each of which is hereby incorporated in its entirety, including all tables, figures, and claims. For example, BNP, which is synthesized in the cardiac ventricles and correlates with left ventricular pressure, amount of dyspnea, and the state of neurohormonal modulation, makes this peptide the first potential marker for heart failure. Measurement of plasma BNP concentration is evolving as a very efficient and cost effective mass screening technique for identifying patients with various cardiac abnormalities regardless of etiology and degree of LV systolic dysfunction that can potentially develop into obvious heart failure and carry a high risk of a cardiovascular event. Finding a simple blood test that would aid in the diagnosis and management of patients with CHF clearly would have a favorable impact on the staggering costs associated with the disease.

In the case of canine BNP, various radioimmunoassays are known in the art. Such radioimmunoassays require that the user deal with various legal requirements for the possession, handling, and use of radioactive materials. In addition, because known assays are competitive assays, the time required to complete an assay may be as long as two days. See, e.g., Protocol for Radioimmunoassay Kit, Phoenix Pharmaceuticals Canine BNP Radioimmunoassay, http://www.phoenixpeptide.com/allobesity/qcdata/RIK/protocol 1-128.html. This extended time period is due presumably to the kinetics of equilibrium of an analyte at pg/mL concentrations in the competitive format.

### SUMMARY OF THE INVENTION

The present invention relates in part to compositions and methods designed to determine the presence or amount of BNP, or its fragments, in a sample. The compositions and methods described herein can meet the need in the art for rapid, sensitive and specific assays for the measurement of BNP and for the diagnosis and prognosis of disease in the veterinary setting.

In a first object, the present invention relates to assay methods configured for the measurement of canine BNP or related fragments. Such assays are preferably sandwich assays using antibodies selected to bind canine BNP, and preferably provide a signal that distinguishes canine BNP from human BNP. Such assays are also preferably nonradioactive in format.

In a related object of the invention, the present invention relates to methods for the diagnosis and/or prognosis of an animal, comprising performing an assay configured to detect the presence or amount of canine BNP (and/or one or more fragments related thereto) in a sample obtained from the animal, and relating the assay result to a particular diagnosis and/or prognosis. In preferred embodiments, the animal is suspected of having or has been diagnosed as having one or more cardiovascular conditions as defined herein.

In various aspects, these methods comprise contacting a sample with a first specific binding member immobilized on a solid phase support, and a second specific binding member conjugated to a detectable label. Following removal of unbound labeled specific binding member from the solid support (e.g., by washing), a signal is detected from detectable label bound to the solid support. These steps are referred to herein as “performing a sandwich assay.” The detected signal may be related to the presence or amount of BNP or related fragments present in the sample.

While the assays of the present invention are configured to bind canine BNP, in preferred embodiments the assays are also configured to distinguish canine BNP from human BNP. An assay is said to “distinguish” two species' BNP if the crossreactivity is less than 1%. Crossreactivity is determined by comparing the slope of an assay signal obtained from one BNP species (e.g., canine) to the assay signal for an equal amount (measured in ng/mL) of another BNP species (e.g., human) between 0 and 0.5 ng/mL. If the signal slope obtained for, in this case, an amount of human BNP is less than 1% of the signal from an equal amount of canine BNP, the assay distinguishes canine from human; that is, the assay does not crossreact with human BNP. If such a signal slope is 1% or more, then the assay would not distinguish canine from human; that is, such an assay crossreacts with human BNP.

In particularly preferred embodiments the assays of the present invention also detect, and preferably do not distinguish, BNP native to at least one other animal species selected from the group consisting of sus (pig), felis (cat), and ovis (sheep). In various embodiments, an assay for canine BNP crossreacts at least 1%, more preferably at least 2%, still more preferably at least 5%, and most preferably at least 10% or more with BNP from sus (pig), felis (cat), and/or ovis (sheep) BNP. Such assays most preferably crossreact less than 1%, and most preferably less than 0.2% with human BNP.

In various embodiments, one or both of the first and second specific binding members used in the assays described herein are antibodies. In preferred embodiments, one or both of the first and second specific binding members do not exhibit substantially identical binding to canine BNP as compared to human BNP, and preferably exhibit a substantially greater affinity for canine BNP than for human BNP. In particularly preferred embodiments, one or both of the first and second specific binding members exhibit substantially identical binding to, and preferably substantially identical affinity for, canine BNP and BNP native to at least one other animal species selected from the group consisting of sus, felis, and ovis.

The assays of the present invention are preferably designed to distinguish various BNP species. An assay is said to “distinguish” between a first group of polypeptides and a second group of polypeptides if the assay provides a signal related to binding of the first group of polypeptides that is at least a factor of 5 greater than a signal obtained from an equal number of molecules of the second group of polypeptides under the same assay conditions, when the assay is performed at no more than twice the amount of the first group of polypeptides necessary to obtain a maximum signal. More preferably, the signal is at least a factor of 10 greater, even more preferably at least a factor of 20 greater, and most preferably at least a factor of 50 greater, at least a factor of 100 greater, or more under such assay conditions.

The term “substantially identical binding” refers to a specific binding member that, when used in an assay, provides signals that are within a factor of 5, and most preferably a factor of 2, for equimolar amounts of two target polypeptides. A factor of 1 indicates that the signals are equal; that signals are within a factor of 2 indicates that one signal is less than or equal to the other signal x 2. Preferably, specific binding members exhibiting substantially identical binding provide signals that are within a factor of about 1.75, more preferably within a factor of about 1.5, still more preferably within a factor of about 1.25, and most preferably within a factor of about 1.1 to 1.

Such specific binding members may also have “substantially identical affinity” with respect to a first target polypeptide and a second target polypeptide, meaning an affinity that is within a factor of 5, and most preferably a factor of 2, for the two target polypeptides. A factor of 1 indicates that the affinities are equal; that affinities are within a factor of 2 indicates that one affinity is less than or equal to the other signal x 2. Preferably, specific binding members exhibiting substantially identical binding provide affinities that are within a factor of about 1.75, more preferably within a factor of about 1.5, still more preferably within a factor of about 1.25, and most preferably within a factor of about 1.1 to 1. A specific binding member has a “substantially greater affinity” for a target polypeptide relative to a non-target polypeptide if the affinity for the target polypeptide is greater than a factor of 5, more preferably greater than a factor of 10, and most preferably greater than a factor of 100 or more than the affinity for the non-target polypeptide.

A signal from an assay is said to “depend upon binding to an antibody” if the antibody participates in formation of a complex necessary to generate the signal. For example, in a sandwich immunoassay formulated using a solid phase antibody and a second antibody conjugate, each of which must bind to an analyte to form the sandwich, each of the solid phase antibody and second antibody participate in formation of the complex necessary to generate the signal.

As described hereinafter, such assays may be designed in a variety of ways known to those of skill in the art. Preferred assays are sandwich immunoassays, although other methods are well known to those skilled in the art (for example, the use of biosensors comprising an integrated analyte receptor and transducer, or the use of natural receptors for natriuretic peptides that are known in the art). Any suitable immunoassay may be utilized, for example, assays which directly detect analyte binding (e.g., by ellipsometric detection), enzyme-linked immunoassays (ELISA), radioimmunoassays (RIAs), competitive binding assays, sandwich immunoassays, and the like.

Direct labels that may be conjugated to specific binding members include fluorescent or luminescent tags, metals, dyes, radionuclides, and the like, attached to the antibody. Indirect labels include various enzymes well known in the art, such as alkaline phosphatase, horseradish peroxidase and the like. Antibodies attached to a second molecule, such as a detectable label, are referred to herein as “antibody conjugates.” The skilled artisan will also understand that natural receptors for the natriuretic peptides exist, and that these receptors may also be used in a manner akin to antibodies in providing binding assays.

Solid phases that may be used to immobilize specific binding members include include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates.

In another object, the present invention relates to monoclonal antibodies that bind to canine BNP, and that are either “insensitive” or “sensitive” to other BNP species. Antibodies are said to be “insensitive” with respect to a first target polypeptide and a second target polypeptide if the antibody exhibits substantially identical binding to the two target polypeptides. Antibodies that are not “insensitive” with respect to two polypeptides are said to be “sensitive” with respect to the polypeptides.

In preferred embodiments, the antibodies of the present invention do not exhibit substantially identical binding to canine BNP as compared to human BNP, and preferably exhibit a substantially greater affinity for canine BNP than for human BNP. In particularly preferred embodiments, the antibodies of the present invention exhibit substantially identical binding to, and preferably substantially identical affinity for, canine BNP and BNP native to at least one other animal species selected from the group consisting of sus, felis, and ovis.

In yet another object, one or more antibodies and/or antibody conjugates of the present invention may be provided as kits for determining the presence or amount of BNP. These kits preferably comprise devices and reagents for performing at least one assay as described herein on a test sample. Such kits preferably contain sufficient reagents to perform one or more such determinations.

The summary of the invention described above is non-limiting and other features and advantages of the invention will be apparent from the following detailed description of the invention, and from the claims.

### BRIEF DESCRIPTION OF THE FIGURES

FIG. 1 shows the sequence of human BNP₇₇₋₁₀₈, and corresponding sequences from selected mammalian species (dog, pig, cat, sheep, and mouse).

FIG. 2 shows box-and-whisker plots obtained from measurement of canine samples with an assay configured to bind canine BNP in various disease states.

FIG. 3 shows the crossreactivity of an exemplary assay for canine BNP (triangles) with porcine (sus) BNP (squares), and an absence of crossreactivity with human BNP (diamonds).

### DETAILED DESCRIPTION OF THE INVENTION

The present invention relates in part to methods and compositions for measuring BNP in non-human animals, in particular using assays configured to bind canine BNP. As described herein, antibodies may be generated that selectively recognize canine BNP (and various degradation products), and used in assays. Such assays can provide important diagnostic and prognostic information in the veterinary setting

The term “natriuretic peptide” as used herein refers to members of a group of naturally occurring polypeptide hormones that act in the body to oppose the activity of the renin-angiotensin system, and their biosynthetic precursors and biologically active fragments. There are three major human natriuretic peptides: atrial natriuretic peptide (ANP), which is synthesized in the atria; brain-type natriuretic peptide (BNP), which is synthesized in the ventricles; and C-type natriuretic peptide (CNP), which is synthesized in the brain.

The human pro-BNP molecule is a 108-amino acid molecule as shown in SEQ ID NO: 1:

| (SEQ ID NO: 1) | |
|---|---|
| HPLGSPGSAS DLETSGLQEQ RNHLQGKLSE LQVEQTSLEP LQESPRPTGV | 50 |
| WKSREVATEG IRGHRKMVLY TLRAPRSPKM VQGSGCFGRK MDRISSSSGL | 100 |
| GCKVLRRH. | 108 |

Mature human BNP (shown underlined above) is a 32 amino acid molecule representing amino acids 77-108 of this precursor, which may be referred to as BNP₇₇₋₁₀₈. The remaining residues 1-76 are referred to hereinafter as NT-proBNP, or BNP₁₋₇₆.

The sequences pro-BNP from various other species, including pigs (Sus scrofa), cows (Bos Taurus), domestic dogs (Canis familiaris), domestic cats (Felis catus), sheep (Ovis aries), mice (Mus musculus), rats (Rattus norvegicus), etc., are known in the art. See, e.g., Liu et al., Gene 292: 183-190, 2002. The canine BNP sequence is also disclosed in U.S. Pat. No. 5,948,761.

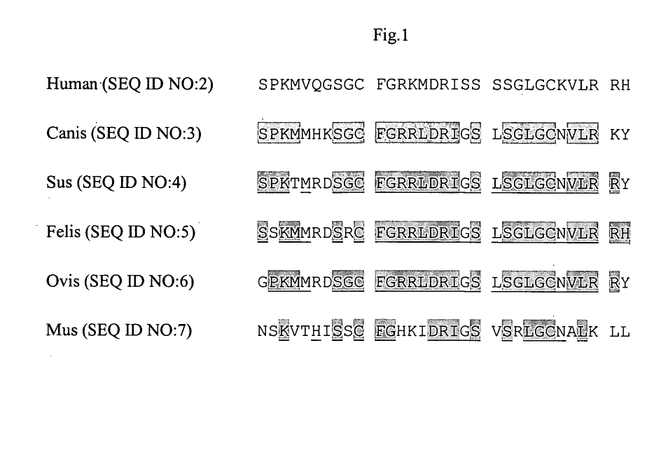

An alignment of the 32 amino acid residues from various non-human species corresponding to the human BNP molecule is shown in FIG. 1. In this figure, shaded regions show BNP residues in various non-human species that are identical with the human BNP sequence, while underlined region show residues that are identical with the canine BNP sequence. As shown in this figure, only single contiguous residues unique in comparison to the human sequence and common amongst non-human BNP species. Despite this extensive homology, the present invention provides methods and compositions that distinguish human and canine BNP.

It is known from studies of human BNP that degradation of the polypeptide can result in BNP-related fragments in blood-derived samples. Failure to consider the fragments that may be present in a clinical sample may have serious consequences for the accuracy of any diagnostic or prognostic method. Consider for example a simple case, where a sandwich immunoassay is provided for BNP, and a significant amount (e.g., 50%) of the BNP that had been present has now been degraded, resulting in a loss of residues from the amino and/or carboxyl terminus. An immunoassay formulated with antibodies that bind a region lost from BNP in producing the fragment(s) may underestimate the amount of BNP originally present in the sample, potentially resulting in a “false negative” result in an assay. Thus, it is preferred that antibodies selected in accordance with the invention for use in sandwich assays not be selected on the basis of particular BNP epitopes, but instead on the basis of clinical results. That is, antibodies may be selected by comparison of assay results obtained from a first “normal” population and a second “diseased” population, selecting for an antibody pair that is able to distinguish these two populations.

The term “fragment” as used herein refers to a polypeptide that comprises at least six contiguous amino acids of a polypeptide from which the fragment is derived, but is less than the complete parent polypeptide. In preferred embodiments, a fragment refers to a polypeptide that comprises at least 10 contiguous amino acids of a polypeptide from which the fragment is derived; at least 15 contiguous amino acids of a polypeptide from which the fragment is derived; or at least 20 contiguous amino acids of a polypeptide from which the fragment is derived. The term “related fragment” as used herein refers to one or more fragments of a particular polypeptide or its biosynthetic parent that may be detected as a surrogate for the polypeptide itself or as independent markers.

The term “solid phase” as used herein refers to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001).

As used herein, the term “purified” in reference to polypeptides (including antibodies) does not require absolute purity. Instead, it represents an indication that the polypeptide(s) of interest is (are) in a discrete environment in which abundance (on a mass basis) relative to other proteins is greater than in a biological sample. By “discrete environment” is meant a single medium, such as a single solution, a single gel, a single precipitate, etc. Purified polypeptides may be obtained by a number of methods including, for example, laboratory synthesis, chromatography, preparative electrophoresis, centrifugation, precipitation, affinity purification, etc. One or more “purified” polypeptides of interest are preferably at least 10% of the protein content of the discrete environment. One or more “substantially purified” polypeptides are at least 50% of the protein content of the discrete environment, more preferably at least 75% of the protein content of the discrete environment, and most preferably at least 95% of the protein content of the discrete environment. Protein content is determined using a modification of the method of Lowry et al., J. Biol. Chem. 193: 265, 1951, described by Hartree, Anal Biochem 48: 422-427 (1972), using bovine serum albumin as a protein standard.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies, monoclonal antibodies, polyclonal antibodies, and antibodies obtained by molecular biological techniques (e.g., by phage display methods) are also included by reference in the term “antibody.” Preferred antibodies specifically bind to a target antigen with a minimum affinity of 10⁹ M⁻¹ to 10¹⁰ M⁻¹.

The term “specifically binds” is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction-bound (r) of labeled ligand at various concentrations (c). The data are graphed using the Scatchard equation: r/c=K(n−r):
where
r=moles of bound ligand/mole of receptor at equilibrium;
c=free ligand concentration at equilibrium;
K=equilibrium association constant; and
n=number of ligand binding sites per receptor molecule
   By graphical analysis, r/c is plotted on the Y-axis versus r on the X-axis thus producing a Scatchard plot. The affinity is the negative slope of the line k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “discrete” as used herein refers to areas of a surface that are non-contiguous. That is, two areas are discrete from one another if a border that is not part of either area completely surrounds each of the two areas. The term “independently addressable” as used herein refers to discrete areas of a surface from which a specific signal may be obtained. One skilled in the art will appreciate that antibody zones can also be independent of each other, but can be in contact with each other on a surface.

The term “test sample” as used herein refers to a sample in which the presence or amount of one or more analytes of interest are unknown and to be determined in an assay, preferably an immunoassay. Preferably, a test sample is a bodily fluid obtained for the purpose of diagnosis, prognosis, or evaluation of a subject, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine and saliva. In addition, one of skill in the art would realize that some test samples would be more readily analyzed following a fractionation or purification procedure, for example, separation of whole blood into serum or plasma components. Preferred samples may be obtained from bacteria, viruses and animals, such as dogs and cats. Particularly preferred samples are obtained from humans. By way of contrast, a “standard sample” refers to a sample in which the presence or amount of one or more analytes of interest are known prior to assay for the one or more analytes.

The term “disease sample” as used herein refers to a tissue sample obtained from a subject that has been determined to suffer from a given disease. Methods for clinical diagnosis are well known to those of skill in the art. See, e.g., Kelley's Textbook of Internal Medicine, 4^{th} Ed., Lippincott Williams & Wilkins, Philadelphia, Pa., 2000; The Merck Manual of Diagnosis and Therapy, 17^{th} Ed., Merck Research Laboratories, Whitehouse Station, N.J., 1999.

The term “about” as used herein refers to +/−10% of a given number.

Use of BNP as a Prognostic And Diagnostic Marker

As noted above, increased blood levels of natriuretic peptides have been found in certain disease states, suggesting a role in the pathophysiology of those diseases, including stroke, congestive heart failure (CHF), cardiac ischemia, systemic hypertension, and acute myocardial infarction. See, e.g., WO 02/089657; WO 02/083913; WO 03/016910; Hunt et al., Biochem. Biophys. Res. Comm. 214: 1175-83 (1995); Venugopal, J. Clin. Pharm. Ther. 26: 15-31, 2001; and Kalra et al., Circulation 107: 571-3, 2003; each of which is hereby incorporated in its entirety, including all tables, figures, and claims. In the case of canines, increased BNP levels are reportedly associated with severity of heart failure.

As also noted above, the failure to consider BNP fragments that may be present in a clinical sample when measuring “BNP” may have serious consequences for the accuracy of any diagnostic or prognostic method. Thus, while the present assays are configured to bind BNP, it will be apparent to the artisan that both BNP and any fragments thereof that retain the binding epitopes used in the sandwich assay will result in a detectable signal from the assays described herein. For convenience, the BNP molecules bound by a particular assay are referred to herein as “BNP-related species.”

Measurement of BNP and its fragments may be applied to the diagnosis and/or prognosis of cardiovascular conditions generally. The term “cardiovascular conditions” refers to a diverse set of disorders of the heart and vasculature, including atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, peripheral vascular disease, and acute coronary syndromes.

The term “diagnosis” as used herein refers to methods by which the skilled artisan can estimate and even determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future event in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient.

The term “correlating,” as used herein in reference to the use of diagnostic and prognostic indicators, refers to comparing the presence or amount of the indicator in a patient to its presence or amount in persons known to suffer from, or known to be at risk of, a given condition; or in persons known to be free of a given condition, i.e. “normal individuals”. For example, a marker level in a patient sample can be compared to a level known to be associated with heart failure generally, or with a specific type of congestive heart failure (e.g., a particular NYHA class; decompensated heart failure; compensated heart failure; etc.). The sample's marker level is said to have been correlated with a diagnosis; that is, the skilled artisan can use the marker level to determine whether the patient suffers from a specific type of CHF, and respond accordingly. Alternatively, the sample's marker level can be compared to a marker level known to be associated with a good outcome (e.g., the absence of near term mortality), such as an average level found in a population of normal individuals.

Selection of Antibodies

The generation and selection of antibodies for use in the methods described herein may be accomplished several ways. For example, one way is to purify fragments or to synthesize the fragments of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with one or more polypeptide(s) of interest (e.g., canine BNP or one of its fragments) and, if required, comparing the results to the affinity and specificity of the antibodies with one or more polypeptide(s) that are desired to be excluded from binding (e.g., human BNP or one of its fragments). The screening procedure can involve immobilization of the purified polypeptides in separate wells of microtiter plates. The solution containing a potential binding moiety (e.g., an antibody or groups of antibodies) is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The binding moieties so identified may then be further analyzed for affinity and specificity to the natriuretic peptide(s) of interest in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody in an assay may be a more important measure than absolute affinity and specificity of an antibody.

Those skilled in the art will recognize that many approaches can be taken in producing antibodies or other binding moieties and screening and selecting for affinity and specificity for the various target polypeptides, but these approaches do not change the scope of the invention.

Assay Measurement Srategies

Numerous methods and devices are well known to the skilled artisan for the detection and analysis of polypeptides or proteins in test samples. In preferred embodiments, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

Preferred assays of the invention are “rapid,” which as used herein refers to an assay in which an assay result is obtained within about 6 hours, more preferably within about 4 hours, still more preferably within about 2 hours, even more preferably within about 1 hour, and most preferably within about 30 minutes, of the addition of sample to the assay.

The use of immobilized antibodies specific for the one or more polypeptides is specifically contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like, by a variety of means known in the art. An assay strip could be prepared by coating the antibody or a plurality of antibodies in an array on solid support. Coupling of the antibody can be direct or indirect (for example, a biotinylated antibody may be immobilized to a surface to which avidin has previously been coupled).

Likewise, the use of antibodies conjugated to a detectable label is also contemplated by the present invention. Biological assays require methods for detection, and one of the most common methods for quantitation of results is to conjugate an enzyme, fluorophore or other molecule to a protein or nucleic acid that has affinity for one of the components in the biological system being studied. Antibody-enzyme conjugates (primary or secondary antibodies) are among the most common protein-protein conjugates used. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels are fluorescent latex particles such as those described in U.S. Pat. Nos. 5,763,189, 6,238,931, and 6,251,687; and International Publication WO95/08772, each of which is hereby incorporated by reference in its entirety. Exemplary conjugation to such particles is described hereinafter.

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulffiydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links.

Heterobifunctional cross-linkers possess two or more different reactive groups that allow for sequential conjugations with specific groups of proteins, minimizing undesirable polymerization or self-conjugation. Heterobifunctional reagents are also used when modification of amines is problematic. Amines may sometimes be found at the active sites of macromolecules, and the modification of these may lead to the loss of activity. Other moieties such as sulfhydryls, carboxyls, phenols and carbohydrates may be more appropriate targets. A two-step strategy allows for the coupling of a protein that can tolerate the modification of its amines to a protein with other accessible groups. A variety of heterobifunctional cross-linkers, each combining different attributes for successful conjugation, are commercially available. Cross-linkers that are amine-reactive at one end and sulfhydryl-reactive at the other end are quite common. If using heterobifunctional reagents, the most labile group is typically reacted first to ensure effective cross-linking and avoid unwanted polymerization.

Many factors must be considered to determine optimum cross-linker-to-target molar ratios. Depending on the application, the degree of conjugation is an important factor. For example, when preparing immunogen conjugates, a high degree of conjugation is normally desired to increase the immunogenicity of the antigen. However, when conjugating to an antibody or an enzyme, a low-to-moderate degree of conjugation may be optimal to ensure that the biological activity of the protein is retained. It is also important to consider the number of reactive groups on the surface of the protein. If there are numerous target groups, a lower cross-linker-to-protein ratio can be used. For a limited number of potential targets, a higher cross-linker-to-protein ratio may be required. This translates into more cross-linker per gram for a small molecular weight protein.

Cross-linkers are available with varying lengths of spacer arms or bridges connecting the reactive ends. The most apparent attribute of the bridge is its ability to deal with steric considerations of the moieties to be linked. Because steric effects dictate the distance between potential reaction sites for cross-linking, different lengths of bridges may be considered for the interaction. Shorter spacer arms are often used in intramolecular cross-linking studies, while intermolecular cross-linking is favored with a cross-linker containing a longer spacer arm.

The inclusion of polymer portions (e.g., polyethylene glycol (“PEG”) homopolymers, polypropylene glycol homopolymers, other alkyl-polyethylene oxides, bis-polyethylene oxides and co-polymers or block co-polymers of poly(alkylene oxides)) in cross-linkers can, under certain circumstances be advantageous. See, e.g., U.S. Pat. Nos. 5,643,575, 5,672,662, 5,705,153, 5,730,990, 5,902,588, and 5,932,462; and Topchieva et al., Bioconjug. Chem. 6: 380-8, 1995). For example, U.S. Pat. No. 5,672,662 discloses bifunctional cross-linkers comprising a PEG polymer portion and a single ester linkage. Such molecules are said to provide a half-life of about 10 to 25 minutes in water.

The analysis of a plurality of polypeptides may be carried out separately or simultaneously with one test sample. For separate or sequential assay, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of polypeptides on a single surface. Particularly useful physical formats comprise surfaces having a plurality of discrete, adressable locations for the detection of a plurality of different analytes. Such formats include protein microarrays (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., one or more polypeptides of the invention) for detection at each location. Surfaces may alternatively comprise one or more discrete particles (e.g., microparticles or nanoparticles) immobilized at discrete locations of a surface, where the microparticles comprise antibodies to immobilize one analyte (e.g., one or more polypeptides of the invention) for detection.

In addition, one skilled in the art would recognize the value of testing multiple samples (for example; at successive time points) from the same individual. Such testing of serial samples will allow the identification of changes in polypeptide levels over time. Increases or decreases in polypeptide levels, as well as the absence of change in such levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of disease having similar symptoms, identification of the severity of the event, identification of the disease severity, and identification of the patient's outcome, including risk of future events.

A panel consisting of the polypeptides referenced above, and optionally including other protein markers useful in diagnosis, prognosis, or differentiation of disease, may be constructed to provide relevant information related to differential diagnosis. Such a panel may be constructed to detect 1, 2, 3, 4, 5, 6, 7, 8, 9, 10, 15, 20, or more or individual analytes, including one or more polypeptides of the present invention. The analysis of a single analyte or subsets of analytes could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single analyte or a subset of analytes in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of analytes could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In certain embodiments, the signal obtained from an assay need not be related to the presence or amount of one or more natriuretic peptide(s); rather, the signal may be directly related to the presence or absence of a disease, or the likelihood of a future adverse outcome related to a disease. For example, a level of signal x may indicate that y pg/mL of a natriuretic peptide is present in the sample. A table may then indicate that y pg/mL of that natriuretic peptide indicates congestive heart failure. It may be equally valid to simply relate a level of signal x directly to congestive heart failure, without determining how much of the natriuretic peptide is present. Such a signal is preferably obtained from an immunoassay using the antibodies of the present invention, although other methods are well known to those skilled in the art.

As discussed above, samples may continue to degrade BNP or fragments thereof, even once the sample is obtained. Thus, it may be advantageous to add one or more protease inhibitors to samples prior to assay. Numerous protease inhibitors are known to those of skill in the art, and exemplary inhibitors may be found in, e.g., The Complete Guide for Protease Inhibition, Roche Molecular Biochemicals, updated Jun. 3, 1999 at http://www.roche-applied-science.com/fst/products.htm?/prod_inf/manuals/protease/prot_toc.htm, and European Patent Application 03013792.1 (published as EP 1 378 242 A1), each of which is hereby incorporated in its entirety. Because various metalloproteases and calcium-dependent proteases are known to exist in blood-derived samples, chelators such as EGTA and/or EDTA, also act as protease inhibitors. In addition, or in the alternative, inhibitors of neutral endopeptidase and/or dipeptidyl peptidase may be used.

In developing diagnostic or prognostic test, data for one or more potential markers may be obtained from a group of subjects. The group of subjects is divided into at least two sets, and preferably the first set and the second set each have an approximately equal number of subjects. The first set includes subjects who have been confirmed as having a disease or, more generally, being in a first condition state. For example, this first set of patients may be those that have recently had a disease incidence, or may be those having a specific type of disease. The confirmation of the condition state may be made through a more rigorous and/or expensive testing such as MRI or CT. Hereinafter, subjects in this first set will be referred to as “diseased”.

The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects. In still another alternative, this second set may represent those at a different time point from disease incidence.

Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

A marker often is incapable of definitively identifying a patient as either diseased or non-diseased. For example, if a patient is measured as having a marker level that falls within the overlapping region of the diseased and non-diseased Gaussian curves, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. ROC curves having an area under the curve of 0.5 indicate complete randomness, while an area under the curve of 1.0 reflects perfect separation of the two sets. Thus, the area under the ROC curve can be used to determine the effectiveness of the test.

Measures of test accuracy may be obtained as described in Fischer et al., Intensive Care Med. 29: 1043-51, 2003; Zhou et al., Statistical Methods in Diagnostic Medicine, John Wiley & Sons, 2002; and Motulsky, Intuitive Biostatistics, Oxford University Press, 1995; and other publications well known to those of skill in the art, and used to determine the effectiveness of a given marker or panel of markers. These measures include sensitivity and specificity, predictive values, likelihood ratios, diagnostic odds ratios, hazard ratios, and ROC curve areas. As discussed above, suitable tests may exhibit one or more of the following results on these various measures:

A ROC curve area of greater than about 0.5, more preferably greater than about 0.7, still more preferably greater than about 0.8, even more preferably greater than about 0.85, and most preferably greater than about 0.9;
a positive or negative likelihood ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less;
an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; and/or
a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less.

Measures of diagnostic accuracy such as those discussed above are often reported together with confidence intervals or p values. These may be calculated by methods well known in the art. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Use of BNP for Determining a Treatment Regimen

A useful diagnostic or prognostic indicator such as BNP can help clinicians select between alternative therapeutic regimens. For example, patients with elevation in cardiac troponin T or I following an acute coronary syndrome appear to derive specific benefit from an early aggressive strategy that includes potent antiplatelet and antithrombotic therapy, and early revascularization. Hamm et al., N. Engl. J. Med. 340: 1623-9 (1999); Morrow et al., J. Am. Coll. Cardiol. 36: 1812-7 (2000); Cannon et al., Am. J. Cardiol. 82: 731-6 (1998). Additionally, patients with elevation in C-reactive protein following myocardial infarction appear to derive particular benefit from HMG-CoA Reductase Inhibitor therapy. Ridker et al., Circulation 98: 839-44 (1998). Among patients with congestive heart failure, pilot studies suggest that ACE inhibitors may reduce BNP levels in a dose dependent manner. Van Veldhuisen et al., J. Am. Coll. Cardiol. 32: 1811-8 (1998).

Similarly, “tailoring” diuretic and vasodilator therapy based on the level of one or more natriuretic peptides may improve outcomes. See, e.g., Troughton et al., Lancet 355: 1126-30 (2000). Finally, in a single pilot study of 16 patients found that randomization to an ACE inhibitor rather than placebo following Q-wave MI was associated with reduced BNP levels over the subsequent 6-month period. Motwani et al., Lancet 341: 1109-13 (1993). Because BNP is a counter-regulatory hormone with beneficial cardiac and renal effects, it is likely that a change in BNP concentration reflects improved ventricular function and reduced ventricular wall stress. A recent article demonstrates the correlation of NT pro-BNP and BNP assays (Fischer et al., Clin. Chem. 47: 591-594 (2001). It is a further objective of this invention that the concentration of natriuretic peptides, either individually or considered in groups of markers, can be used to guide diuretic and vasodilator therapy to improve patient outcome. Additionally, the measurement of natriuretic peptides, either individually or considered in groups of markers, for use as a prognostic indicator for patients is within the scope of the present invention.

Recent studies in patients hospitalized with congestive heart failure suggest that serial BNP measurements may provide incremental prognositic information as compared to a single measurement; that is, assays can demonstrate an improving prognosis when BNP falls after therapy than when it remains persistently elevated. Cheng et al., J. Am. Coll. Cardiol. 37: 386-91 (2001). Thus, serial measurements of natriuretic peptides according to the present invention may increase the prognostic and/or diagnostic value of a marker in patients, and is thus within the scope of the present invention.

### EXAMPLES

The following examples serve to illustrate the present invention. These examples are in no way intended to limit the scope of the invention.

### Example 1

### Blood Sampling

Blood is preferably collected by venous puncture using a 20 gauge multi-sample needle and evacuated tubes, although fingertip puncture, plantar surface puncture, earlobe puncture, etc., may suffice for small volumes. For whole blood collection, blood specimens are collected by trained study personnel in EDTA-containing blood collection tubes. For serum collection, blood specimens are collected by trained study personnel in thrombin-containing blood collection tubes. Blood is allowed to clot for 5-10 minutes, and serum is separated from insoluble material by centrifugation. For plasma collection, blood specimens are collected by trained study personnel in citrate-containing blood collection tubes and centrifuged for ≧12 minutes. Samples may be kept at 4° C. until use, or frozen at −20° C. or colder for longer term storage. Whole blood is preferably not frozen.

### Example 2

### Recombinant Antibody Preparation

Immunization of Mice with Antigens and Purification of RNA From Mouse Spleens

Two species of mice can be used for immunization: Balb/c (Charles River Laboratories, Wilmington, Mass.) and A/J (Jackson Laboratories, Bar Harbor, Me.). Each of ten mice were immunized intraperitoneally with antigen using 50 μg protein in adjuvant (e.g., Freund's complete or Quil A) on day 0, 14, and 28. Tests bleeds of mice were obtained through puncture of the retro-orbital sinus. The mice were subsequently boosted with 50 μg of protein on days 42 and 43, or on days 42, 56, 57, and 59.

On days 45 (first boost schedule) and 59 (second boost schedule), the spleens were harvested, macerated, and the spleen suspension pulled through an 18 gauge needle until viscous and all cells are lysed, then transferred to a microcentrifuge tube. The sample was divided evenly between two microcentrifuge tubes and the following added in order, with mixing by inversion after each addition: 100 μl 2 M sodium acetate (pH 4.0), 1.0 ml water-saturated phenol (Fisher Scientific, Pittsburgh, Pa.), 200 μl chloroform/isoamyl alcohol 49:1 (Fisher Scientific, Pittsburgh, Pa.). The solution was vortexed for 10 seconds and incubated on ice for 15 min. Following centrifugation at 14,000 rpm for 20 min at 2-8° C., the aqueous phase was transferred to a fresh tube. An equal volume of water saturated phenol/chloroform/isoamyl alcohol (50:49:1) was added, and the tube vortexed for ten seconds. After a 15 min incubation on ice, the sample was centrifuged for 20 min at 2-8° C., and the aqueous phase transferred to a fresh tube and precipitated with an equal volume of isopropanol at −20° C. for a minimum of 30 min. Following centrifugation at 14,000 rpm for 20 min at 4° C., the supernatant was aspirated away, the tubes briefly spun and all traces of liquid removed.

The resulting RNA pellets were each dissolved in 300 μl of solution D, combined, and precipitated with an equal volume of isopropanol at −20° C. for a minimum of 30 min. The sample was centrifuged 14,000 rpm for 20 min at 4° C., the supernatant aspirated as before, and the sample rinsed with 100 μl of ice-cold 70% ethanol. The sample was again centrifuged 14,000 rpm for 20 min at 4° C., the 70% ethanol solution aspirated, and the RNA pellet dried in vacuo. The pellet was resuspended in 100 μl of sterile distilled water, and the RNA stored at −80° C.

Preparation of Complementary DNA (cDNA)

The total RNA purified as described above was used directly as template for preparation of cDNA. RNA (50 μg) was diluted to 100 μL with sterile water, and 10 μL-130 ng/mL oligo dTI₂ is added. The sample was heated for 10 min at 70° C., then cooled on ice. 40 μL 5× first strand buffer was added (Gibco/BRL, Gaithersburg, Md.), 20 μL 0.1 M dithiothreitol (Gibco/BRL, Gaithersburg, Md.), 10 μL 20 mM deoxynucleoside triphosphates (dNTP's, Boehringer Mannheim, Indianapolis, Ind.), and 10 μL water on ice. The was then incubated at 37° C. for 2 min. 10 μL reverse transcriptase (Superscript II, Gibco/BRL, Gaithersburg, Md.) was added and incubation continued at 37° C. for 1 hr. The cDNA products are used directly for polymerase chain reaction (PCR).

Amplification of cDNA by PCR

To amplify substantially all of the H and L chain genes using PCR, primers were chosen that corresponded to substantially all published sequences. 33 oligonucleotides are synthesized to serve as 5′ primers for the H chains, and 29 oligonucleotides are synthesized to serve as 5′ primers for the kappa L chains, substantially as described in U.S. Pat. No. 2,003,0104477. Amplification by PCR was performed separately for each pair of 5′ and 3′ primers. A 50 μL reaction was performed for each primer pair with 50 pmol of 5′ primer, 50 pmol of 3′ primer, 0.25 μL Taq DNA Polymerase (5 units/μL, Boehringer Mannheim, Indianapolis, Ind.), 3 μL cDNA (described in Example 2), 5 μL 2 mM dNTP's, 5 μL 10×Taq DNA polymerase buffer with MgCl2 (Boehringer Mannheim, Indianapolis, Ind.), and H₂O to 50 μL. The dsDNA products of the PCR process were then subjected to asymmetric PCR using only 3′ primer to generate substantially only the anti-sense strand of the target genes.

Purification of ss-DNA by High Performance Liquid Chromatography and Kinasing ss-DNA

The H chain ss-PCR products and the L chain ss-PCR products were ethanol precipitated by adding 2.5 volumes ethanol and 0.2 volumes 7.5 M ammonium acetate and incubating at −20° C. for at least 30 min. The DNA was pelleted by centrifuging in an Eppendorf centrifuge at 14,000 rpm for 10 min at 2-8° C. The supernatant was carefully aspirated, and the tubes briefly spun a 2nd time. The last drop of supernatant was removed with a pipet. The DNA was dried in vacuo for 10 min on medium heat. The H chain and L chain products were pooled separately in 210 μL water. The ss-DNA was purified by high performance liquid chromatography (HPLC), and the ss-DNA eluted from the HPLC collected in 0.5 min fractions. Fractions containing ss-DNA were ethanol precipitated, pelleted and dried as described above. The dried DNA pellets were pooled in 200 μL sterile water.

If desired, the ss-DNA was kinase-treated on the 5′ end in preparation for mutagenesis. 24 pL 10× kinase buffer (United States Biochemical, Cleveland, Ohio), 10.4 μL 10 mM adenosine-5′-triphosphate (Boehringer Mannheim, Indianapolis, Ind.), and 2 μL polynucleotide kinase (30 units/μL, United States Biochemical, Cleveland, Ohio) was added to each sample, and the tubes incubated at 37° C. for 1 hr. The reactions were stopped by incubating the tubes at 70° C. for 10 min. The DNA was purified with one extraction of equilibrated phenol (pH>8.0, United States Biochemical, Cleveland, Ohio)-chloroform-isoamy-1 alcohol (50:49:1) and one extraction with chloroform:isoamyl alcohol (49:1). After the extractions, the DNA was ethanol precipitated and pelleted as described above.

Antibody Phage Display Vector

The antibody phage display vector contained the DNA sequences encoding the heavy and light chains of a mouse monoclonal Fab fragment inserted into a vector substantially as described by Huse, WO 92/06024. To make the first derivative cloning vector, deletions were made in the variable regions of the H chain and the L chain by oligonucleotide directed mutagenesis (Kunkel, Proc. Natl. Acad. Sci. USA 82:488 (1985); Kunkel, et al., Methods. Enzymol. 154:367 (1987)). These mutations delete the region of each chain from the 5′ end of CDR1 to the 3′ end of CDR3, and add a DNA sequence where protein translation would stop. The resulting cloning vector is called BS 11.

Changes were made to BS11 to generate the cloning vector used in the present screening methods. The amber stop codon between the heavy chain and the pseudo gene VIII sequence was removed so that every heavy chain is expressed as a fusion protein with the gene VIII protein. A HindIII restriction enzyme site in the sequence between the 3′ end of the L chain and the 5′ end of the alkaline phosphatase signal sequence was deleted. The interchain cysteine residues at the carboxyl-terminus of the L and H chains were changed to serine residues. Nonessential DNA sequences on the 5′ side of the lac promoter and on the 3′ side of the pseudo gene VIII sequence were deleted. A transcriptional stop DNA sequence was added to the vector at the L chain cloning site. Finally, DNA sequences for protein tags were added to different vectors to allow enrichment for polyvalent phage by metal chelate chromatography or by affinity purification using a decapeptide tag and a magnetic latex having an immobilized antibody that binds the decapeptide tag.

Transformation of E. coli by Electroporation

Electrocompetent E. coli cells were thawed on ice. DNA was mixed with 20-40 μL electrocompetent cells by gently pipetting the cells up and down 2-3 times, being careful not to introduce air-bubbles. The cells were transferred to a Gene Pulser cuvette (0.2 cm gap, BioRAD, Hercules, Calif.) that has been cooled on ice, again being careful not to introduce an air-bubble in the transfer. The cuvette was placed in the E. coli Pulser (BioRAD, Hercules, Calif.) and electroporated with the voltage set at 1.88 kV according to the manufacturer's recommendation. The transformed sample was immediately diluted to 1 ml with 2×YT broth.

### Example 3

### Preparation of Biotinylated Antigens and Antibodies

Protein antigens or antibodies were dialyzed against a minimum of 100 volumes of 20 mM borate, 150 mM NaCl, pH 8 (BBS) at 2-8° C. for at least 4 hr. The buffer was changed at least once prior to biotinylation. Protein antigens or antibodies were reacted with biotin-XX-NHS ester (Molecular Probes, Eugene, Oreg., stock solution at 40 mM in dimethylformamide) at a final concentration of 1 mM for 1 hr at room temperature. After 1 hr, the protein antigens or antibodies were extensively dialyzed into BBS to remove unreacted small molecules.

### Example 4

### Preparation of Alkaline Phosphatase-Antigen Conjugates

Alkaline phosphatase (AP, Calzyme Laboratories, San Luis Obispo, Calif.) was placed into dialysis versus a minimum of 100 volumes of column buffer (50 mM potassium phosphate, 10 mM borate, 150 mM NaCl, 1 mM MgSO₄, pH 7.0) at 2-8° C. for at least four hr. The buffer was changed at least twice prior to use of the AP. The AP was diluted to 5 mg/mL with column buffer. The reaction of AP and succinimidyl 4-(N-maleimidomethyl) cyclohexane-1-carboxylate (SMCC, Pierce Chemical Co., Rockford, Ill.) was carried out using a 20:1 ratio of SMCC:AP. SMCC was dissolved in acetonitrile at 20 mg/mL and diluted by a factor of 84 when added to AP while vortexing or rapidly stirring. The solution was allowed to stand at room temperature for 90 min before the unreacted SMCC and low molecular weight reaction products were separated from the AP using gel filtration chromatography (G50 Fine, Pharmacia Biotech, Piscataway, N.J.) in a column equilibrated with column buffer.

Protein antigen was dialyzed versus a minimum of 100 volumes of 20 mM potassium phosphate, 4 mM borate, 150 mM NaCl, pH 7.0 at 2-8° C. for at least four hr. The buffer was changed at least twice prior to use of the antigen. The reaction of antigen and N-succinimidyl 3-[2-pyridyldithio]propionate (SPDP, Pierce Chemical Co., Rockford, Ill.) was carried out using a 20:1 molar ratio of SPDP:antigen. SPDP was dissolved in dimethylformamide at 40 mM and diluted into the antigen solution while vortexing. The solution was allowed to stand at room temperature for 90 min, at which time the reaction was quenched by adding taurine (Aldrich Chemical Co., Milwaukee, Wis.) to a final concentration of 20 mM for 5 min. Dithiothreitol (Fisher Scientific, Pittsburgh, Pa.) was added to the protein at a final concentration of 1 mM for 30 min. The low molecular weight reaction products were separated from the antigen using gel filtration chromatography in a column equilibrated in 50 mM potassium phosphate, 10 mM borate, 150 mM NaCl, 0.1 mM ethylene diamine tetraacetic acid (EDTA, Fisher Scientific, Pittsburgh, Pa.), pH 7.0.

The AP and antigen were mixed together in an equimolar ratio. The reaction was allowed to proceed at room temperature for 2 hr. The conjugate was diluted to 0.1 mg/mL with block containing 1% bovine serum albumin (from 30% BSA, Bayer, Kankakee, Ill.), 10 mM Tris, 150 mM NaCl, 1 mM MgCl₂, 0.1 mM ZnCl₂, 0.1% polyvinyl alcohol (80% hydrolyzed, Aldrich Chemical Co., Milwaukee, Wis.), pH 8.0.

### Example 5

### Preparation of Peptide Conjugates with Keyhole Limpet Hemocyanin and Bovine Serum Albumin

Keyhole Limpet Hemocyanin (KLH) conjugates were made essentially as described in Example 21 of U.S. Pat. No. 6,057,098 with the following modifications: KLH-SMCC was reacted with a 2-fold excess of peptide thiol consisting of 90% specific cysteine containing peptide and 5% each of PADRE peptide having a cysteine at the N-terminus of the peptide and the C-terminus of the peptide (peptide 1024.03 from Alexander et al., Immunity 1: 751-761, 1994).

Bovine Serum Albumin (BSA) conjugates with peptide were made essentially as described in Example 21 of U.S. Pat. No. 6,057,098. The BSA-biotin peptide conjugates were made by first biotinylating the BSA (Example 9 of U.S. Pat. No. 6,057,098), then conjugating with peptide.

### Example 6

### Preparation of Avidin Magnetic Latex

Magnetic latex (Estapor, 10% solids, Bangs Laboratories, Fishers, Ind.) was thoroughly resuspended and 2 ml aliquoted into a 15 ml conical tube. The magnetic latex was suspended in 12 ml distilled water and separated from the solution for 10 min using a magnet. While still in the magnet, the liquid was carefully removed with a 10 mL sterile pipet. This washing process was repeated three times. After the final wash, the latex was resuspended in 2 ml of distilled water. In a separate 50 ml conical tube, 10 mg of avidin-HS (NeutrAvidin, Pierce, Rockford, Ill.) was dissolved in 18 ml of 40 mM Tris, 0.15 M sodium chloride, pH 7.5 (TBS). While vortexing, the 2 ml of washed magnetic latex was added to the diluted avidin-HS and the mixture vortexed an additional 30 seconds. This mixture was incubated at 45° C. for 2 hr, shaking every 30 minutes. The avidin magnetic latex was separated from the solution using a magnet and washed three times with 20 ml BBS as described above. After the final wash, the latex was resuspended in 10 ml BBS and stored at 4° C.

Immediately prior to use, the avidin magnetic latex was equilibrated in panning buffer (40 mM TRIS, 150 mM NaCl, 20 mg/mL BSA, 0.1% Tween 20 (Fisher Scientific, Pittsburgh, Pa.), pH 7.5). The avidin magnetic latex needed for a panning experiment (200 μl/sample) was added to a sterile 15 ml centrifuge tube and brought to 10 ml with panning buffer. The tube was placed on the magnet for 10 min to separate the latex. The solution was carefully removed with a 10 mL sterile pipet as described above. The magnetic latex was resuspended in 10 mL of panning buffer to begin the second wash. The magnetic latex was washed a total of 3 times with panning buffer. After the final wash, the latex was resuspended in panning buffer to the initial aliquot volume.

### Example 7

### Enrichment of Polyclonal Phage

Enrichment of Polyclonal Phage Specific to Canine BNP

The first round antibody phage generally was prepared as described in Example 7 of U.S. Pat. No. 6,057,098 from RNA isolated from mice immunized with canine BNP conjugated to KLH and optionally PADRE. The antibody phage samples were panned with avidin magnetic latex generally as described in Example 16 of U.S. Pat. No. 6,057,098. The first two rounds of antibody phage samples were selected with canine BNP conjugated to BSA-biotin (1×10⁻⁸ M final BSA concentration), in the presence of 10⁻⁶ M final concentration BSA-SMCC to compete away antibodies specific to the SMCC arm. Selections were continued for three additional rounds with canine BNP conjugated to BSA-biotin (1×10⁻⁹ M final BSA concentration) and 10⁻⁶ M final concentration BSA-SMCC. The antibody phage sample was subcloned into a plasmid expression vector generally as described in Example 18 of U.S. Pat. No. 6,057,098.

Canine BNP Carboxyl Terminus-Specific antibodies

The first round antibody phage were generally prepared as described in Example 7 of U.S. Pat. No. 6,057,098 from RNA isolated from mice immunized with canine BNP₁₃₄₋₁₄₀ (CNVLRKY, numbered in accordance with Swiss-Prot accession number P16859) conjugated to KLH and optionally PADRE. The antibody phage samples were panned with avidin magnetic latex generally as described in Example 16 of U.S. Pat. No. 6,057,098. The first round antibody phage samples (10 samples from 5 different spleens) were selected with canine BNP₁₃₄₋₁₄₀ BSA-biotin at 1×10⁻⁸ M final concentration BSA and 10⁻⁶ M final concentration BSA-SMCC. The BSA-SMCC was added to remove antibodies specific to the SMCC arm. The eluted phage were enriched with 7F11 magnetic latex, then the phage samples were panned a second time as described above except canine BNP₁₃₄₋₁₄₀ BSA-biotin at 1×10⁻⁹ M final concentration BSA was used. The phage samples eluted from the 2^{nd} round of panning were pooled, and the third round of panning was done as described above with the pooled phage. The antibody phage sample was subcloned into a plasmid expression vector generally as described in Example 18 of U.S. Pat. No. 6,057,098.

### Example 8

### Biochemical Analyses

BNP is measured using standard sandwich immunoassay techniques using one canine BNP-specific antibody paired with a second antibody specific to the canine BNP carboxyl terminus as described in the previous example. One antibody is biotinylated using N-hydroxysuccinimide biotin (NHS-biotin) at a ratio of about 5 NHS-biotin moieties per antibody. The biotinylated antibody is then added to wells of a standard avidin 384 well microtiter plate, and biotinylated antibody not bound to the plate is removed. This formed an anti-BNP solid phase in the microtiter plate. The second aritibody is conjugated to alkaline phosphatase using standard techniques, using SMCC and SPDP (Pierce, Rockford, Ill.). The immunoassays are performed on a TECAN Genesis RSP 200/8 Workstation. Test samples (10 μL) are pipeted into the microtiter plate wells, and incubated for 60 min. The sample is then removed and the wells washed with a wash buffer, consisting of 20 mM borate (pH 7.42) containing 150 mM NaCl, 0.1% sodium azide, and 0.02% Tween-20. The alkaline phosphatase-antibody conjugate is then added to the wells and incubated for an additional 60 min, after which time, the antibody conjugate is removed and the wells washed with a wash buffer. A substrate, (AttoPhos®, Promega, Madison, Wis.) is added to the wells, and the rate of formation of the fluorescent product is related to the concentration of the BNP in the test samples.

The BNP-related species bound by the canine BNP assays (pg/ml) were measured in canine populations divided into three diagnosis groups: dyspnea, asymptomatic heart failure, and normal. Simple statistics are list in table 1:

TABLE 1

| | | Mini- | Maxi- | | | Std | |
|---|---|---|---|---|---|---|---|
| Diagnosis | N | mum | mum | Mean | Median | Dev | CV |
| Asymptomatic | 43 | 0.82 | 24.56 | 5.44 | 3.29 | 5.59 | 102.79 |
| Dyspnea | 62 | 0.82 | 280.23 | 27.50 | 12.81 | 48.73 | 177.22 |
| Normal | 25 | 0.45 | 8.92 | 2.28 | 1.92 | 1.77 | 77.67 |

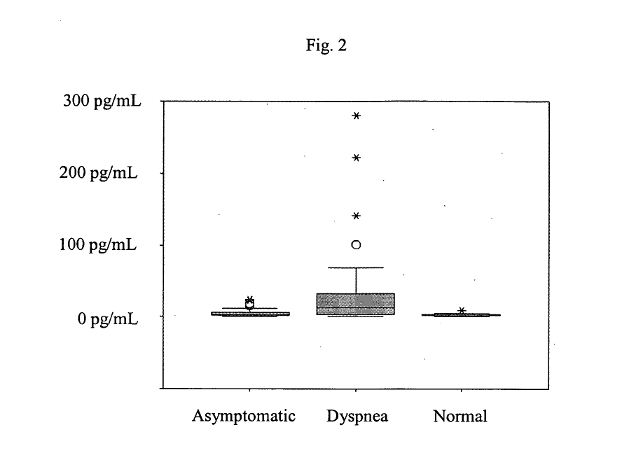

The results are displayed graphically in box-and-whisker format in FIG. 2. These data are significantly different in median (Kruskal-Wallis Test p<0.0001); BNP measurement within Dyspnea diagnosis group are the highest among three groups, and Asymptomatic diagnosis group have higher BNP level than normal diagnosis group as well (P=0.001).

Using ROC curve analysis, the three diagnosis groups were compared pairwise, as shown in Table 2:

TABLE 2

| Comparison | ROC curve area |
|---|---|
| normal v. asymptomatic | 0.74 |
| normal v. dyspnea | 0.81 |
| normal v. diseased† | 0.78 |

†diseased = dyspnea + asymptomatic heart failure

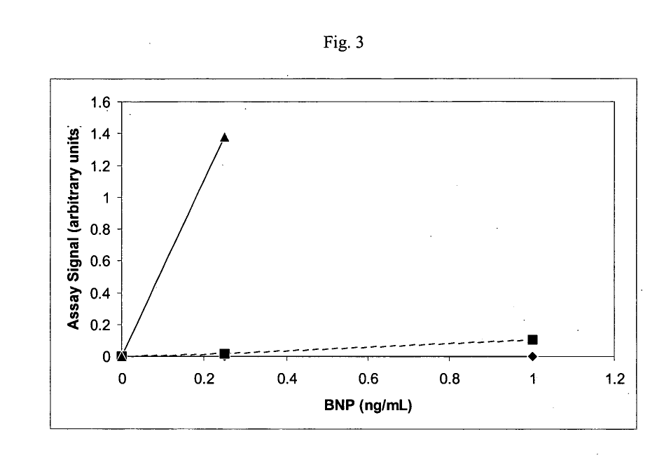

The minimum detectable level of canine BNP in this assay was 0.87 pg/mL. Cross-reactivity with human BNP was assessed using a sample that measured ≧1000 pg/mL using the BIOSITE® TRIAGE® human BNP test. This sample gave a result of 5 pg/mL in the canine-specific test. No crossreactivity was observed with human BNP (FIG. 3, compare triangles to diamonds). In contrast, the assay crossreacted about 2% with porcine (sus) BNP (FIG. 3, compare triangles to squares).

Assays that distinguish or that do not distinguish between BNP species can find particular use in studies or therapies where xenospecific BNP is administered. For example, comparative studies of the clearance of human BNP often make use of experimental animals. In such studies, the amount of human BNP present in a sample may be determined by measuring the total BNP using an assay that does not distinguish between BNP species, and subtracting the contribution from non-human BNP using an assay that does distinguish between such species. Likewise, if the non-human BNP is used in humans therapeutically, an assay that distinguishes between such BNP species may be used to monitor the therapeutic dose.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for determining the presence or amount of BNP-related species in a non-human animal, comprising:
performing a sandwich assay on a sample obtained from said non-human animal, wherein said assay is configured to bind canine BNP, and wherein the results of said sandwich assay are indicative of the presence or amount of said BNP-related species in said sample.

2. A method according to claim 1, wherein said sandwich assay comprises contacting the sample with a first antibody immobilized on a solid support, and a second antibody conjugated to a detectable label.

3. A method according to claim 2, wherein one or both of said first and second antibodies are monoclonal antibodies.

4. A method according to claim 1, wherein said assay is configured to distinguish canine BNP from human BNP.

5. A method according to claim 4, wherein said assay is configured to not distinguish canine BNP from BNP native to one or more species selected from the group consisting of sus, felis, and ovis.

6. A method according to claim 1, wherein said non-human animal is a canine.

7. A method according to claim 1, wherein said non-human animal is a feline.

8. A method according to claim 2, wherein one or both of said first or second antibodies exhibit a substantially greater affinity for canine BNP than for human BNP.

9. A method according to claim 1, wherein the assay is a rapid assay.

10. A method according to claim 1, wherein the assay provides a result within about 1 hour of contacting said sample with said first or second antibody.

11. A method according to claim 1, wherein said assay employs nonradioactive detection.

12. A method according to claim 1, wherein said assay employs fluorescent detection.

13. A method according to claim 1, wherein said assaying step comprises performing mass spectrometry.

14. A method according to claim 1, further comprising relating the presence or amount of said BNP-related species in said sample to the diagnosis or prognosis of a cardiovascular condition.

15. A method according to claim 14, wherein said cardiovascular condition is heart failure.

16. A method according to claim 14, wherein said prognosis is death.

17. A monoclonal antibody that binds to canine BNP with a substantially greater affinity for canine BNP than human BNP.

18. A monoclonal antibody according to claim 17, wherein said antibody is a recombinant antibody.

19. A monoclonal antibody according to claim 17, wherein said antibody is insensitive with respect to canine BNP and BNP native to one or more species selected from the group consisting of sus, felis, and ovis.

20. A kit for determining the presence or amount of BNP-related species in a non-human animal, comprising:
a first antibody that binds canine BNP immobilized on a solid phase; and
a second antibody that binds canine BNP conjugated to a detectable label;
each in an amount sufficient to perform at least one sandwich immunoassay on a sample.


SEQUENCE LISTING (8 sequences)

SEQ ID NO: 1 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 32 aa, Homo sapiens
SPKMVQGSGCFGRKMDRISSSSGLGCKVLRRH

SEQ ID NO: 3 - protein, 32 aa, Canis familiaris
SPKMMHKSGCFGRRLDRIGSLSGLGCNVLRKY

SEQ ID NO: 4 - protein, 32 aa, Sus scrofa
SPKTMRDSGCFGRRLDRIGSLSGLGCNVLRRY

SEQ ID NO: 5 - protein, 32 aa, Felis catus
SSKMMRDSRCFGRRLDRIGSLSGLGCNVLRRH

SEQ ID NO: 6 - protein, 32 aa, Ovis aries
GPKMMRDSGCFGRRLDRIGSLSGLGCNVLRRY

SEQ ID NO: 7 - protein, 32 aa, Mus musculus
NSKVTHISSCFGHKIDRIGSVSRLGCNALKLL

SEQ ID NO: 8 - protein, 7 aa, Canis familiaris
CNVLRKY